(19) United States
(12) Patent Application Publication
(10) Pub. No.: US 2026/0172040 A1
(43) Pub. Date: Jun. 18, 2026

(54) LOW-LEVEL CURRENT SIGNAL GENERATOR

(71) Applicants: Tao Shen, Kent, OH (US); Jinsai Cheng, Kent, OH (US)

(72) Inventors: Tao Shen, Kent, OH (US); Jinsai Cheng, Kent, OH (US)

(21) Appl. No.: 19/420,552

(22) Filed: Dec. 15, 2025

Related U.S. Application Data
(60) Provisional application No. 63/733,750, filed on Dec. 13, 2024.

(51) Int. Cl. H03L 7/24

(52) U.S. Cl. CPC H03L 7/24

(57) ABSTRACT

Signal generator circuits including a microcontroller for receiving a command signal, a function generator for outputting a waveform output, and a current stimulator circuit comprising a current divider portion and a current regulator portion, wherein the current divider portion is coupled to the function generator and receives the waveform output, and wherein the current regulator portion includes an output current, and further methods for applying a current having a targeted waveform to a load including the steps of providing a load having an impedance, providing a signal generator circuit coupled to the load, and commanding the signal generator to supply the targeted waveform to the load via the command signal to thereby apply the current having the targeted waveform to the load, wherein the command signal includes the parameters of the targeted waveform.

### CROSS-REFERENCE TO RELATED APPLICATIONS

The present application claims the benefit of U.S. Provisional Patent Application No. 63/733,750, filed on Dec. 13, 2024, which is incorporated herein in its entirety.

### STATEMENT REGARDING FEDERALLY SPONSORED RESEARCH OR DEVELOPMENT

This invention was made with government support under Grant No. awarded 1R42AG078721-01 titled, “Developing a clinical diagnostic tool for age-related cochlear synaptopathy” by the National Institute of Health. The government has certain rights in the invention.

### FIELD OF THE INVENTION

Embodiments of the present invention provide signal generators capable of generating a variety waveform patterns at different frequencies and with nanoampere-level currents which may then be used to induce microvolt-level signals in a load.

### BACKGROUND OF THE INVENTION

Signal generators are devices that can produce various waveforms with different frequencies and amplitudes. Signal generators are widely used in experimental, teaching, production, and research environments. Currently, the frequency synthesis theory of signal generators primarily encompasses four technologies: direct analog frequency synthesis, phase-locked frequency synthesis, direct digital frequency synthesis, and hybrid frequency synthesis.

A low-level current generator is one type of signal generator that is able to generate current signals having micro and nano level currents, such as microampere and nanoampere currents, and may later be used to induce a voltage with an amplitude in the range of microvolts. Such low level current signal generators are widely used in industries such as biomedical, oil exploration, earthquake detection, and the detection of rock bursts in geophysical environments. For example, in the biomedical discipline, signals involved in EEG (Electroencephalogram) studies are usually very weak, in the range of 1 to 160 uV amplitude and thus need nanoampere level current waveforms to induce such voltage signals. Prior art signal generators utilized a 50 uV standard signal to calibrate an EEG signal, and this micro-voltage level signal requires a nano-ampere level current generator to induce. The existing current waveform signal generators in the market are expensive, bulky, and cannot reach nano-ampere level operation. As such there is need for an improved signal generator capable of producing nanoampere current signals.

Additionally, some applications of signal generators only need pulses instead of continuous waveform signals to complete their operation. There is an urgent need for low-level current pulse generators to further the development of high-level technology in many areas. Thus, there is a need for signal generators capable of supplying nano-ampere level current for both continuous and pulse signals.

### SUMMARY OF THE INVENTION

One or more embodiments of the present invention provide a signal generator circuit including a microcontroller including an input interface for receiving a command signal and an output interface, a function generator including an input interface coupled to the output interface of the microcontroller, and wherein the function generator further includes a waveform output, and a current stimulator circuit comprising a current divider portion and a current regulator portion, wherein the current divider portion is coupled to the function generator and receives the waveform output, and wherein the current regulator portion includes an output current.

Another embodiment of the present invention provides a signal generator circuit as described in any of the foregoing embodiments and further wherein the current regulator portion further includes an operational amplifier (op-amp) which produces the output current.

Another embodiment of the present invention provides a signal generator circuit as described in any of the foregoing embodiments and further wherein the op-amp is in an inverted configuration.

Another embodiment of the present invention provides a signal generator circuit as described in any of the foregoing embodiments and further wherein the current regulator portion further includes a load impedance resistor coupled to the load and the op-amp.

Another embodiment of the present invention provides a signal generator circuit as described in any of the foregoing embodiments and further wherein the current divider portion comprises a resistor network.

Another embodiment of the present invention provides a signal generator circuit as described in any of the foregoing embodiments and further wherein the resistor network reduces a magnitude of the current of the waveform output from the function generator by a factor of 500 or greater.

Another embodiment of the present invention provides a signal generator circuit as described in any of the foregoing embodiments and further wherein the resistor network reduces a magnitude of the current of the waveform output from the function generator by a factor of 1000 or greater.

Another embodiment of the present invention provides a signal generator circuit as described in any of the foregoing embodiments and further wherein the resistor network is arranged to provide the current regulator portion a reduced current waveform of from 1 nanoamperes to 25 nanoamperes.

Another embodiment of the present invention provides a signal generator circuit as described in any of the foregoing embodiments and further wherein the resistor network is arranged to provide the current regulator portion a reduced current waveform of from 1 nanoamperes to 10 nanoamperes.

Another embodiment of the present invention provides a signal generator circuit as described in any of the foregoing embodiments and further wherein the output current has a magnitude of from 1 nanoamperes to 25 nanoamperes.

Another embodiment of the present invention provides a signal generator circuit as described in any of the foregoing embodiments and further wherein the output current has a magnitude of from 1 nanoamperes to 10 nanoamperes.

Yet further embodiments of the present invention provide a method of applying a current having a targeted waveform to a load, the method including the steps of providing a load having an impedance, providing the signal generator circuit as in any embodiment above, coupling the output current to the load, and commanding the signal generator to supply the targeted waveform to the load via the command signal to thereby apply the current having the targeted waveform to the load, wherein the command signal includes the parameters of the targeted waveform.

### BRIEF DESCRIPTION OF THE DRAWINGS

FIG. 1 is a circuit diagram of a signal generator according to an embodiment of the present invention;

FIG. 2 is a circuit diagram of a current divider according to an embodiment of the present invention;

FIG. 3 is a flow chart that illustrates a control flow of a microcontroller according to an embodiment of the present invention;

FIG. 4 is a photographic view of a signal generator according to an embodiment of the present invention;

FIGS. 5A and 5B are photographic views of oscilloscope measured waveforms generated by a signal generator according to an embodiment of the present invention; and

FIGS. 6A and 6B are graphs that illustrate the measured amplitude of square-wave and sine-wave waveforms applied to a load at varying input currents.

### DETAILED DESCRIPTION OF ILLUSTRATIVE EMBODIMENTS

### Introduction

The terms “coupled to” or “couples with” (and the like) as used herein without further qualification are intended to describe either an indirect or direct electrical connection. Thus, if a first device “couples” to a second device, that connection can be through a direct electrical connection where there are only parasitics in the pathway, or through an indirect electrical connection via intervening items including other devices and connections. For indirect coupling, the intervening item generally does not modify the information of a signal but may adjust its current level, voltage level, and/or power level.

Embodiments of the invention are based, at least in part, on the discovery of a current signal generator based on Direct Digital Synthesis (DDS) technology. DDS methods may be used in frequency synthesis to create arbitrary waveforms from a single, fixed-frequency reference clock. While the embodiments disclosed herein utilize DDS methods for signal generation and function generators, other applications of DDS technology include, without limitation, local oscillators in communication systems, mixers, modulators, sound synthesizers and as part of a digital phase-locked loop.

Those of ordinary skill in the art understand a function generator as a device or integrated circuit that is used to generate several types of electrical waveforms over a wide range of frequencies. Some of the most common waveforms produced by the function generator are the sine wave, square wave, triangular wave, and sawtooth shapes. These waveforms can be either repetitive or a single-shot/pulse. Embodiments of the signal generators according to the present invention are capable of generating various waveform patterns at different frequencies and nanoampere-level currents. Such nanoampere-level currents may then be used to induce microvolt-level signals.

### Signal Generators

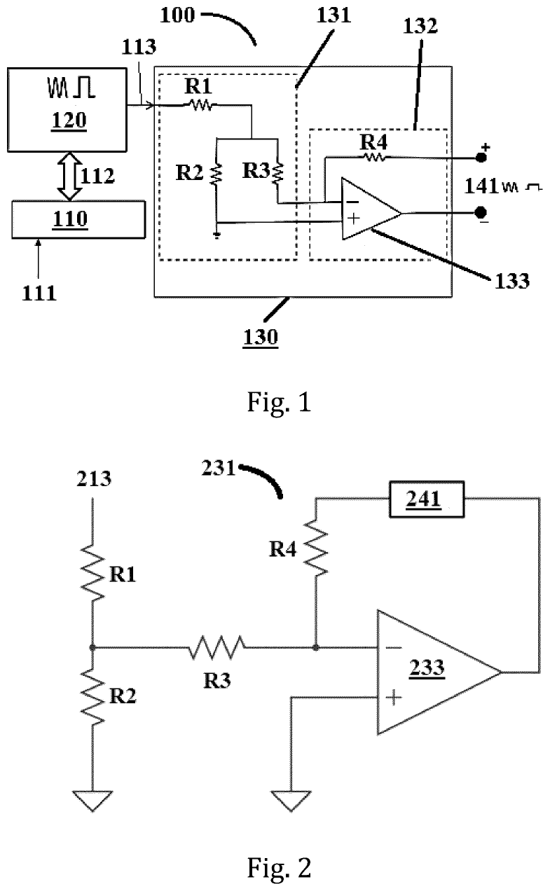

An exemplary embodiment of a signal generator 100, which may also be referred to as a low-level signal generator, is shown in FIG. 1. The integrated circuit comprising signal generator 100 includes a microcontroller 110 which is coupled to function generator 120, which may also be referred to as a waveform generator, which is coupled to current stimulator 130 where current stimulator 130 includes current divider portion 131 coupled to current regulator portion 132, and further the current regulator portion may be coupled to a load 141 which receives the generated current signal and waveform. During operation of the signal generator, a command signal 111 is sent to microcontroller 110 and includes information regarding the configuration of the target waveform to be generated. Microcontroller 110 processes the command signal and communicates with function generator 120 via coupling interface 112. Function generator 120 produces the commanded waveform and directs the signal to current stimulator 130 where the waveform first passes through current divider portion 131 including resistors R1, R2, and R3, then proceeding to current regulator portion 132 including operation amplifier (op-amp) 133, load 141, and resistor R4. Load 141 receives the waveform signal, which may be measured using other equipment external to signal generator 100.

In one or more embodiments, signal generators may be powered by an external power source (not shown). The power source may be coupled with the microcontroller and function generator to enable both to be operational.

Signal generators according to embodiments of the present invention are not particularly limited in terms of the loads that are coupled to the current stimulator, and in particular the current regulator portion of the current stimulator. Suitable loads to be coupled with signal generators according to embodiments of the present invention include, without limit, equipment requiring calibration that is used in industries such as biomedical, oil exploration, earthquake detection, and the detection of rock bursts in geophysical environments.

In one or more embodiments, signal generators may produce a continuous waveform subjected to the load. In these and other embodiments, signal generators may produce a pulse waveform subjected to the load. In these and other embodiments, the pulse waveform may include a plurality of pulses with a time delay between pulses. The time delay between pulses may be further variable according to the desired calibration being performed using the signal generator. Signal generators according to yet further embodiments of the present invention may be capable of producing both continuous and pulse waveforms subjected to the load.

Signal generators according to embodiments of the present invention are not particularly limited in the types of waveforms, regardless of whether the waveform is continuous or pulses, which may be generated. Non-limiting examples of waveforms capable of being supplied by signal generators according to the present invention include one or more of sine wave, square wave, triangle wave, and sawtooth wave.

In one or more embodiments, signal generators according to the present invention are configured to provide to a load, any of the waveforms described above having a current of 100 nanoamperes or less, in other embodiments 50 nanoamperes or less, in other embodiments 25 nanoamperes or less, in other embodiments 20 nanoamperes or less, in other embodiments 15 nanoamperes or less, in other embodiments 10 nanoamperes or less, in other embodiments 9 nanoamperes or less, in other embodiments 8 nanoamperes or less, in other embodiments 7 nanoamperes, in other embodiments 6 nanoamperes or less, in other embodiments 5 nanoamperes or less, in other embodiments 4 nanoamperes or less, in other embodiments 3 nanoamperes or less, in other embodiments 2 nanoamperes or less, and in other embodiments 1 nanoamperes or less.

In one or more embodiments, signal generators according to the present invention are configured to provide to a load, any of the waveforms described above having a current of from 1 nanoampere to 100 nanoamperes, in other embodiments from 1 nanoampere to 50 nanoamperes, in other embodiments from 1 nanoampere to 25 nanoamperes, in other embodiments from 1 nanoampere to 20 nanoamperes, in other embodiments from 1 nanoampere to 15 nanoamperes, in other embodiments from 1 nanoampere to 10 nanoamperes, in other embodiments from 1 nanoampere to 9 nanoamperes, in other embodiments from 1 nanoampere to 8 nanoamperes, in other embodiments from 1 nanoampere to 7 nanoamperes, in other embodiments from 1 nanoampere to 6 nanoamperes, in other embodiments from 1 nanoampere to 5 nanoamperes, in other embodiments from 1 nanoampere to 4 nanoamperes, in other embodiments from 1 nanoampere to 3 nanoamperes, and in other embodiments from 1 nanoampere to 2 nanoamperes.

### Microcontrollers

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

All of these and similar terms are to be associated with the appropriate physical quantities and are merely convenient labels applied to these quantities. Unless specifically stated otherwise, or as is apparent from the discussion, terms such as “processing” or “computing” or “calculating” or “determining” or “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

Embodiments of low-level signal generators according to the present invention generally comprise a microcontroller. Microcontrollers suitable for use in the present invention may include any commercially available processor, microprocessor, microcontroller, controllers including programmable sub-systems such as those available from Arduino, Texas Instruments, Freescale Kinetis, STMicroelectronics STM32, Intel, AMD, and the like. The controllers may also be a System on a Chip (SoC) such as a Raspberry Pi and the like. In these and other embodiments, the microcontroller is configured to receive input commands for controlling an output signal to the function generator. In addition to including one or more processor(s), microprocessor(s), microcontroller(s), or controller(s), microcontrollers suitable for use in the present invention may further comprise well-known circuits that provide functionality to the CPU such as a user interface, clock circuits, network communications, cache, power supplies, I/O circuits, and the like. In some embodiments, the user interface comprises a keypad, electronic buttons, speaker, touchscreen, display, camera, microphone, or other user interaction mechanisms. In these and other embodiments, the microcontroller is further configured to manage the frequency, waveform, and cycles of an output signal to the function generator.

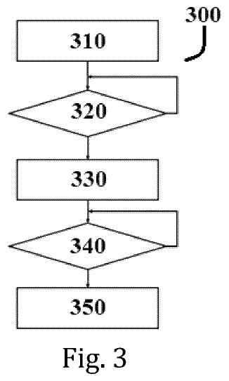

An exemplary control flow of 300 signal generators according to the present invention is shown in FIG. 3. As shown in FIG. 3, control flow 300 is initialized at step 310 where the microcontroller sets up the frequency and the waveform components to be outputted to the function generator. At step 320, the microcontroller either receives an input command signal including a preset duration, tₛ, and proceeds to step 330 or further awaits receipt of an input command signal. At step 330, the microcontroller sends the output signal, including the frequency and waveform components to the function generator such that the function generator outputs a waveform to a load via the current stimulator. In step 340, when the duration of the input command, t, exceeds the preset duration included in the command signal, tₛ, the microcontroller proceeds to step 350 and stops sending the output signal.

In one or more embodiments control flows for signal generators according to the present invention may include further steps. Such further steps may include verification steps that the waveform being output to the load is within an acceptable tolerance of the commanded waveform. Such verification steps may include attaching current sensors to the load that are coupled to the microcontroller to compare measured current versus commanded current. The person of ordinary skill in the art understands that further diagnostic steps may be included.

### Function Generators

The person of ordinary skill in the art readily understands that a function generator is an electronic device that produces electrical waveforms over a wide range of frequencies. Simple function generators usually generate triangular waveform whose frequency can be controlled smoothly as well as in steps. This triangular wave is used as the basis for all of its other outputs. The triangular wave is generated by repeatedly charging and discharging a capacitor from a constant current source. This produces a linearly ascending and descending voltage ramp. As the output voltage reaches upper or lower limits, the charging or discharging is reversed using a comparator, producing the linear triangle wave. By varying the current and the size of the capacitor, different frequencies may be obtained. Sawtooth waves can be produced by charging the capacitor slowly with low current but using a diode over the current source to discharge quickly—the polarity of the diode changes the polarity of the resulting sawtooth, i.e., slow rise and fast fall, or fast rise and slow fall.

In one or more embodiments, the function generator is configured to generate a waveform based upon the output signal from the microcontroller. In these and other embodiments, the function generator includes an input interface for receiving commands from the microcontroller and a waveform output coupled to the current stimulator. In these and other embodiments the microcontroller and function generator each include a serial peripheral interface for communication, wherein the microcontroller issues commands to the function generator via the microcontroller's output serial peripheral interface and the function generator's input interface. The function generator processes or otherwise responds to the command signal from the microcontroller to produce the targeted waveform including the commanded waveform characteristics including the frequency and waveform components. The current supplied by the function generator in the waveform output is tunable to change the output current of the signal generator applied to the load.

### Current Stimulators

The current stimulator is configured to receive the waveform from the function generator and reduce the current to nanoampere-levels while maintaining the waveform. As mentioned above, in one or more embodiments of signal generators the current stimulator comprises

An exemplary circuit for a current stimulator 231 suitable for use in signal generators according to the present invention is shown in FIG. 3. As discussed above, the current divider is configured to reduce the current of the waveform provided by the function generator to nanoampere magnitudes while maintaining the waveform characteristics provided by the microcontroller. Waveform output 213 from the function generator serves as a voltage source and resistors R1 and R2 serve as a voltage divider. The resistor, R3, is added to reduce the current to nanoampere-level. Together, R1, R2, and R3 serve as the current divider portion of current stimulator 231. Current stimulator 231 further includes an operational amplifier (Op-amp) 233 in an inverted configuration to maintain the waveform characteristics including the nanoampere level current that is coupled to load 241 and resistor R4. Op-amp 233 and R4 function as a current regulator of current stimulator 231 such that any impedance changes due to changes in load 241 do not adversely impact the quality of the waveform current applied to load 241.

The selection of R4 is made according to the Op-amp's operating range, which thereby provides for the current signal to be reduced to nanoampere level, maintaining the provided waveform even if the impedance of the load changes. In the inverted configuration, as Vᵢₙ increases, Vₒᵤₜ will increase in accordance with the differential gain. However, as Vₒᵤₜ increases, that output voltage is fed back to the inverting input, thereby decreasing the voltage differential between inputs, which acts to bring the output down. What will happen for any given voltage input is that the op-amp will output a voltage very nearly equal to Vᵢₙ, but just low enough so that there is enough voltage difference left between Vᵢₙ and the (−) input to be amplified to generate the output voltage. The relationship between the input voltage and the output voltage may be expressed as follows (equation 1), including the gain, G:

$${V}_{out} = \frac{{V}_{i n}}{1 + ( \frac{1}{G} )} \qquad ( 1 )$$

Accordingly, in this configuration, the op-amp functions as a current source for the load.

In one or more embodiments, current dividers of current stimulators include resistors R1, R2, and R3. In these and other embodiments, the resistance values of R1, R2, and R3 are selected according to the current to be provided at the load. In these and other embodiments, the resistance values of R1, R2, and R3 are selected to provide a nanoampere current value for the load. In such embodiments the resistor network reduces the current from to the function generator to by a factor of 500 or greater, in other embodiments by a factor of 600 or greater, in other embodiments by a factor of 700 or greater, in other embodiments by a factor of 800 or greater, in other embodiments by a factor of 900 or greater, in other embodiments by a factor of 1000 or greater, in other embodiments by a factor of 1500 or greater, in other embodiments by a factor of 2000 or greater. In these and other embodiments, current regulators of current stimulators include an Op-amp and resistor R4 to be a current regulator portion of current stimulators. In these and other embodiments, the current stimulator is coupled to the function generator via the current divider portion and further coupled to the load via the current regulator portion.

### Methods of Applying a Current Waveform to a Load

Embodiments of signal generators according to the present invention may be useful in applying current waveforms with known properties to a load in order to measure a response by the load. As discussed above, the arrangement of the current stimulator portion of signal generators according to the present invention is provided such that the waveforms are maintained, at nanoampere current amplitudes, regardless of the load that the current waveform is applied to. Accordingly, the embodiments of signal generators disclosed herein may be useful for the applications discussed in the background of the invention.

The process for applying a current waveform to a load includes providing a load, providing a signal generator according to any embodiment described herein, preparing a command signal including information related to the waveform characteristics, sending the command signal to the microcontroller of the signal general circuit whereby the signal generator provides an output current to the load, wherein the output current has the waveform characteristics included in the command signal.

In order to demonstrate the practice of the present invention, the following examples have been prepared and evaluated. The examples should not, however, be viewed as limiting the scope of the invention.

### EXAMPLES

### Calibration Pulse Signal Generator

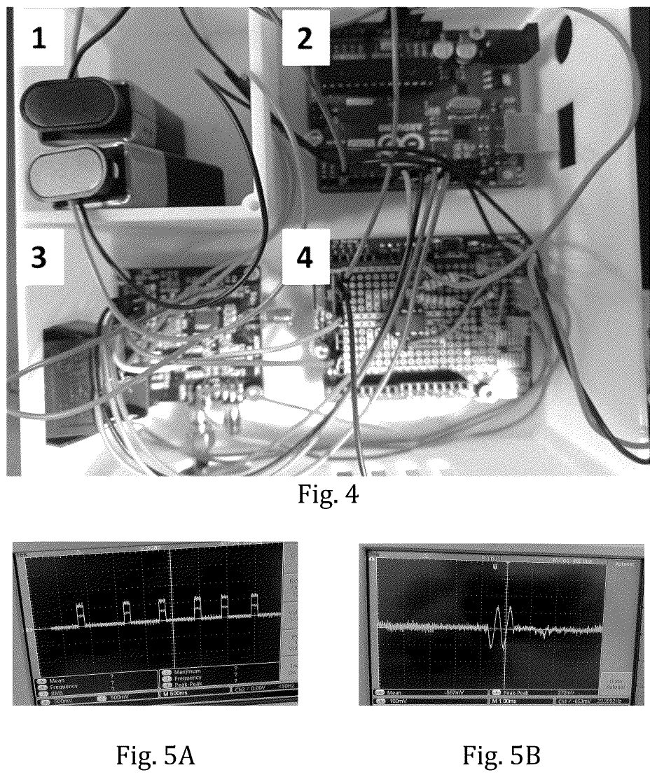

An embodiment of a signal generator according to the present invention was constructed and is shown in FIG. 4. The signal generator of FIG. 4 includes a power source (1), a microcontroller (2), a waveform generator (3), and a current stimulator (4). An Arduino® Uno (Arduino.cc, Torino, Italy) was used as the microcontroller that communicates with the peripheries of the integrated circuit through an SPI (Serial Peripheral Interface). The microcontroller received TTL (transistor-transistor logic) input signal time-locked to other testing equipment instructions, triggering the waveform generator, and defining the output waveform parameters, including waveform type, frequency, timing, and duration. Based on the commands from the microcontroller, the waveform generator produced the desired waveform, including one or more of a square wave pulse or sine wave pulse, which is then directed to the current stimulator. The current stimulator was comprised of two parts: a current divider and a current regulator. The current divider attenuated the current from the waveform generator, while the current regulator maintained a constant current for the waveform, thus eliminating any potential impact from the stimulating loads. The current output of this device could be adjusted over a range of 1 to 10 nA, inclusive, the Arduino is used as the microcontroller that receives the TTL signal from the TDT system and triggers the output of the waveform generator through the SPI. The current divider reduces the current to 1/1000th of the operational amplifier (Op-amp) output. The Calibration Pulse Signal Generator was implemented with recording capabilities by splicing the positive and negative poles of the circuit to the differential preamplifier using splitter cables. A custom configuration file was created using the Tucker Davis Technologies (TDT) SigGenRz software (Tucker Davis Technologies; Alachua, FL, USA) that synchronized the pulse onset to a tested parameter and recording window through a TDT RZ6 Multi-I/O Processor.

As shown in FIGS. 5A and 5B, the Calibration Pulse Signal Generator generated square and sinusoidal current pulses. These pulses were loaded on a 100M ohm resistor. The signals induced by the current pulses were measured by an oscilloscope, and the results are shown in FIG. 4. As the magnitudes of the signals are in 100-millivolt level, thus the actually generated currents are confirmed to be at the nano-ampere level.

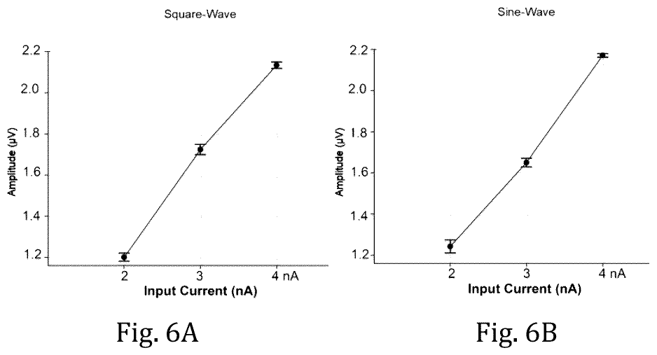

Further testing of the Calibration Pulse Signal Generator was performed, including applying the current waveforms to electrodes in saline solution. The results of this test are reported in FIGS. 6A and 6B, which include the results for square-wave and sine-wave testing, respectively. The Calibration Pulse Signal Generator generated output currents of 2, 3, and 4 nanoamperes (nA). The pulse recording was repeatedly acquired three times over a span of 10 min at each constant current. Standard deviation ranged from 0.01 to 0.038 μV for both signals. The amplitude curve exhibits linear-like growth for both wave types.

Various modifications and alterations that do not depart from the scope and spirit of this invention will become apparent to those skilled in the art. This invention is not to be duly limited to the illustrative embodiments set forth herein.

What is claimed is:

1. A signal generator circuit comprising:
a microcontroller including an input interface for receiving a command signal and an output interface;
a function generator including an input interface coupled to the output interface of the microcontroller, and wherein the function generator further includes a waveform output; and
a current stimulator circuit comprising a current divider portion and a current regulator portion, wherein the current divider portion is coupled to the function generator and receives the waveform output, and wherein the current regulator portion includes an output current.

2. The signal generator circuit of claim 1, wherein the current regulator portion further includes an operational amplifier (op-amp) which produces the output current.

3. The signal generator circuit of claim 2, wherein the op-amp is in an inverted configuration.

4. The signal generator circuit of claim 3, wherein the current regulator portion further includes a load impedance resistor coupled to the load and the op-amp.

5. The signal generator circuit of claim 4, wherein the current divider portion comprises a resistor network.

6. The signal generator circuit of claim 5, wherein the resistor network reduces a magnitude of the current of the waveform output from the function generator by a factor of 500 or greater.

7. The signal generator circuit of claim 5, wherein the resistor network reduces a magnitude of the current of the waveform output from the function generator by a factor of 1000 or greater.

8. The signal generator circuit of claim 5, wherein the resistor network is arranged to provide the current regulator portion a reduced current waveform of from 1 nanoamperes to 25 nanoamperes.

9. The signal generator circuit of claim 5, wherein the resistor network is arranged to provide the current regulator portion a reduced current waveform of from 1 nanoamperes to 10 nanoamperes.

10. The signal generator circuit of claim 1, wherein the output current has a magnitude of from 1 nanoamperes to 25 nanoamperes.

11. The signal generator circuit of claim 1, wherein the output current has a magnitude of from 1 nanoamperes to 10 nanoamperes.

12. A method for applying a current having a targeted waveform to a load, the method comprising the steps of:
providing a load having an impedance;
providing the signal generator circuit of claim 1;
coupling the output current to the load; and
commanding the signal generator to supply the targeted waveform to the load via the command signal to thereby apply the current having the targeted waveform to the load, wherein the command signal includes the parameters of the targeted waveform.
